(19) United States
(12) Patent Application Publication
(10) Pub. No.: US 2023/0248268 A1
(43) Pub. Date: Aug. 10, 2023

(54) Camera-based Respiratory Triggered Medical Scan

(71) Applicant: Siemens Healthcare GmbH, Erlangen (DE)

(72) Inventors: Ruhan Sa, Monmouth Junction, NJ (US); Vivek Singh, Princeton, NJ (US); Ankur Kapoor, Plainsboro, NJ (US)

(21) Appl. No.: 17/649,935

(22) Filed: Feb. 4, 2022

(51) Int. Cl. A61B 5/113; A61B 5/00; A61B 5/11; A61B 5/01; A61B 5/08

(52) U.S. Cl. CPC A61B 5/113; A61B 5/0077; A61B 5/1128; A61B 5/01; A61B 5/7264; A61B 5/704; A61B 5/08

(57) ABSTRACT

Camera-based respiratory triggered medical scanning is provided. A camara captures an image or images of a patient. A processor detects breathing from the camera image or images. Detected breathing is used to automatically trigger the medical scan. An extra breathing sensor is not needed. Manual monitoring is not relied upon.

### BACKGROUND

The present embodiments relate to respiratory motion artifacts in medical scanning. Respiratory motion artifacts degrade the image quality of medical imaging scans or negatively impact treatment scans. For example, in radiation therapy, the breathing motion of the patient can alter the target location during the treatment session. The mis-localization of the target object, such as a tumor, not only affects the treatment of the disease, but also exposes the surrounding healthy tissue to unnecessary radiation. As another example, in lung computed tomography (CT) scans, breathing motion can cause motion artifacts in the CT images, which prevent adequate visualization of the anatomy.

To minimize such uncertainties, breathing instructions are provided during some medical exams. Due to non-compliance of the patient and/or operator differences, mis-registrations still occur. Some patients are not able to hold their breath for extended amounts of time. To overcome these issues, technicians continuously monitor the patient during the scan using a real-time video stream. More advanced sensor-based technologies may assist the technician's monitoring effort by tracking the patient's body surface. An infrared tracking camera and reflective marker block placed on patient may be used to show the breathing to the technician. The surface of the patient may be tracked to predict the breathing for viewing by the technician. A thermal camera or CT may be used to assist in monitoring patient movement. These approaches rely on inconsistent monitoring by the technician.

Apart from manual monitoring or vocal instructions, respiratory-triggered medical scans are also proposed. In one example, in radiation therapy applications, Deep Inspiration Breathing Hold (DIBH) and Cone beam CT images are used to make sure the organs are aligned with the reference planning CT images. As another example, respiratory triggered MRI provides better image quality than breathing-hold based scan or free-breathing scan. Similarly, breathing-triggered lung CT scans have shown significantly less respiratory misregistration artifacts compared to ECG triggered only. These breathing-triggered medical scans rely on breathing specific sensors, such as a pressure-sensing belt, to monitor the breathing motion. These extra breathing sensors add additional steps to the scanning workflow and might not be appropriate in cases such as emergency room scans.

### SUMMARY

Systems, methods, and non-transitory computer readable media with stored instructions are provided for camera-based respiratory triggered medical scanning. A camara captures an image or images of a patient. A processor detects breathing from the camera image or images. Detected breathing is used to automatically trigger the medical scan. An extra breathing sensor is not needed. Manual monitoring is not relied upon.

In a first aspect, a method is provided for camera-based respiratory triggered medical scanning. A camera captures an image of an outer surface of a patient. A processor detects breathing cycle information for the patient from the image and controls a medical scanner with the breathing cycle information.

Different types of cameras may be used. For example, a depth camera captures the image. Depth information from the depth camera is used to detect the breathing. As another example, an infrared or thermal camera captures the image. Temperature information at a head of the patient is captured as the outer surface.

The camera may be positioned at various locations. For example, the camera captures the image while connected to the medical scanner. More than one camera may be used to capture images, such as capture multiple images with multiple cameras. The breathing is detected from the multiple images.

In one embodiment, detecting includes determining values of features from the image and detecting the breathing cycle information from the values. In another embodiment, a region of interest in the image is identified, a feature value for the region of interest in the image is extracted, and the breathing cycle information is detected from the feature value.

The breathing information may include breathing phase and/or magnitude. For example, the breathing cycle information is the phase of the breathing cycle as inhalation, exhalation, or breath hold. The control includes triggering the medical scanner to scan only during one of inhalation, exhalation, or breath hold. The breathing cycle information represents a current state of breathing or is a prediction of breathing at a future time.

In one embodiment, the breathing cycle information is an output of a machine-learned model in response to input of the image to the machine-learned model. In other embodiments, a hand-coded algorithm is used to detect.

In another embodiment, the control of the medical scanner triggers scanning by the medical scanner.

In some embodiments, the capturing, detecting, and controlling are performed free of input from a breath sensor and free of input from a breathing sensor.

In a second aspect, a medical scanner includes a camera configured to image a patient; an image processor configured to detect breathing of the patient from camera data from the camera; a transmitter or receiver configured to scan the patient; and a controller configured to trigger scanning of the patient by the transmitter or receiver, timing of the trigger based on the detected breathing.

In one embodiment, the camera is a depth camera, thermal camera, or infrared camera, and the transmitter or receiver is an imager or a therapy device.

In another embodiment, the image processor is configured to detect the breathing as a breathing cycle phase. The controller is configured to turn on and/or off the transmitter or receiver such that the scan occurs only during inhalation, exhalation, or breath hold.

In yet another embodiment, the image processor is configured to detect the breathing by identification of a region of interest, extraction of a value for a feature of the region of interest from the camera data, and detection of the breathing from the value.

As another embodiment, the image processor is configured to detect by application of a machine-learned model to the camera data.

In a third aspect, a method is provided for camera-based respiratory triggered medical scanning. A camera captures an image of a patient. A processor detects breathing phase or magnitude for the patient from the image. A medical scanner is triggered to scan at a time based on the breathing phase or magnitude.

In one embodiment, images are captured over time with the camera being a depth camera or an infrared camera. The breathing phases at different times are detected based on a region of interest. The medical scanner is triggered to scan only during inhalation, exhalation, or breath hold based on the breathing phases at the different times.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 is a flow chart diagram of one embodiment of a method for camera-based respiratory triggered medical scanning;

FIG. 2 is an example embodiment of the method of FIG. 1;

FIG. 3 illustrates an example depth image represented in grayscale;

FIG. 4 illustrates one embodiment for detecting breathing information with a camera;

FIG. 5 is a flow chart diagram of one embodiment of a method for the breathing detection illustrated in FIG. 4;

FIG. 6 is a block diagram of one embodiment of a system for camera-based respiratory triggered medical scanning.

### DETAILED DESCRIPTION OF EMBODIMENTS

Camera-based respiratory triggered medical scanners provide automatic medical scanning with reduced motion artifacts. Camera-based breathing triggered medical scans automatically control the image acquisition cycle based on breathing pattern and minimize the artifacts caused by respiratory misregistration. The automatic detection and automatic triggering is provided without adding extra steps to the current workflow and reduces the manual monitoring effort. Processor-based detection of the breathing pattern based on a camera may alleviate the need for having a breathing sensor placed on the patient, which can be a limitation when it comes to emergency room cases.

In one embodiment, automatic detection of patient breathing patten uses camera sensors and automatic triggering of the medical scan uses the breathing pattern. Data is captured from a plurality of camera sensors in a medical scanning environment. The data is processed to detect the patient breathing pattern. The breathing pattern is used to trigger scan signals. Upon receiving the scan signal, the medical scanner starts the acquisition cycle or radiation cycle.

The camera-based solution can generalize to different imaging modalities or treatment. Compared to other monitoring-based approaches, the breathing triggered medical scan may alleviate the need for manual determination of image acquisition cycle, reducing inter and intra technician difference and intra and inter patient differences. For imaging, the image quality is improved through minimizing breathing triggered motion artifacts. The need for breathing training and patient breathing monitoring may be eliminated. Radiation therapy may more likely be applied to the planned location and not to other locations without technician's manual monitoring and triggering.

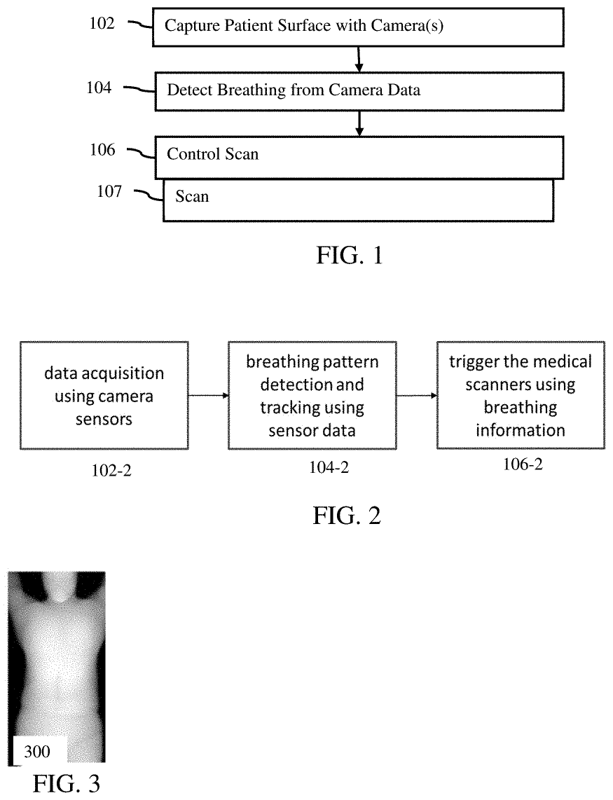

FIG. 1 is a flow chart diagram of one embodiment of a method for camera-based respiratory triggered medical scanning. The camera captures an image or images of the patient. A processor automatically detects breathing of the patient and controls a medical scanner to scan based on the breathing. FIG. 2 shows one embodiment of the method of FIG. 1. Capturing, detecting, and controlling are performed free of input from a breath sensor and breathing sensor. Manual monitoring of the breathing is not needed.

The method of FIG. 1 is performed by a medical imaging system, such as an image processor, camera, and/or scanner. The camera captures the patient surface. An image processor detects breathing from the camera data. The image processor or other controller controls the scan by the scanner based on the detected breathing. In one embodiment, the system of FIG. 6 performs the method, but other systems may be used.

Additional, different, or fewer acts may be provided. For example, act 107 is not performed. As another example, acts for configuring the scanner to scan and/or placing the patient for image capture are provided. Act 106 may be performed without act 107.

The acts are performed in the order shown (top to bottom or numerical) or another order. For example, act 107 is performed as part of act 106 and may begin prior to act 106.

In act 102, a camera captures an image of the patient. An outer surface of a patient is captured as an image. One or more patient images, including patient surface data, are captured. In act 102-2, camera data is acquired using camera sensors.

The camera captures the image as a two-dimensional distribution of pixels, such acquiring red, green, blue (RGB) values. Other information, such as depth information, may be captured as or as part of the image. Thermal, infrared, or another image may be captured.

In one embodiment, the camera is a depth sensor, such as a 2.5D or RGBD (RGB plus depth) sensor (e.g., Microsoft Kinect 2 or ASUS Xtion Pro). The depth sensor may directly measure depths, such as using time-of-flight, interferometry, or coded aperture. The depth sensor may be a camera or cameras capturing a grid projected onto the patient, and a processor reconstructs the outer surface from the structured light in the image or images. The sensor may be multiple cameras capturing 2D images from different directions, allowing reconstruction of the outer surface from multiple images without transmission of structured light. Other optical or non-ionizing sensors may be used, such as a LIDAR camera.

In other embodiments, the camera is an infrared camera or captures optical information at other wavelengths. The camera may be a thermal camera. Infrared or another thermal camera may show temperature as part of the image.

The camera or cameras may be placed in various locations in the room, including attached or connected to the medical scanner itself. The sensor is positioned on a wall, ceiling, or elsewhere in the imaging suite or operating room, such as on a boom generally above the patient. For example, cameras are fixed or attached to the ceiling of the scan room or operating room; on the scanner body, such as at the gantry of the scanner; inside a bore of the scanner; on an extended arm of the scanner, such as on Cone Beam CT scanner during radiation therapy; and/or on a radiation therapy scanner.

The camera (camera sensor) is directed at a patient. The camera's field of view covers at least part of the patient's body during medical image acquisition or during radiation treatment. The camera captures the outer surface of the patient from one or more perspectives. Any portion of the outer surface may be captured, such as the entire patient viewed from one side from head to toe and hand to hand or just the torso. The camera captures the outer surface with the patient in a particular position, such as capturing a front facing surface as the patient lies in a bed or on a table for treatment or imaging.

The outer surface is the skin of the patient. In other embodiments, the outer surface includes clothing. The sensor may use a frequency that passes through clothing and detects skin surface. Alternatively, the outer surface is the clothing.

The camera outputs the sensed pixels and/or depths. The measurements of the outer surface from the camera are camera or surface data for the patient. FIG. 3 shows an example image 300 from surface data where the intensity in grayscale is mapped to the sensed depth. Alternatively, the sensor measurements are processed to determine the outer surface information, such as stereoscopically determining the outer surface from camera images from different angles with image processing.

The surface data is used at the resolution of the sensor. For example, the surface data is at 256×256 pixels. Other sizes may be used, including rectangular fields of view. The surface data may be filtered and/or processed. For example, the surface data is altered to a given resolution. As another example, the surface data is down sampled, such as reducing 256×256 to 64×64 pixels. Each pixel may represent any area, such as each pixel as down sampled to 64×64 representing 1 cm² or greater. Alternatively, the sensor captures at this lower resolution. The surface data may be cropped, such as limiting the field of view. Both cropping and down sampling may be used together, such as to create 64×64 channel data from 256×312 or other input channel data.

In another approach, the surface data is normalized prior to input. The surface data is rescaled, resized, warped, or shifted (e.g., interpolation). The surface data may be filtered, such as low pass filtered.

More than one camera may be used. For example, cameras are positioned at different locations to capture camera data of the outer surface of the patient from different perspectives. The images for any given time from the different cameras may be used separately, such as where the different cameras capture with different types of cameras (e.g., RGBD and thermal cameras). In other embodiments, the camera data (images) from the different cameras are combined, providing a scene. The input from one or more camera sensors may be processed to obtain a desired result stream. For example, point clouds from multiple RGBD cameras are aligned based on known spatial relationship between the fixed cameras and combined into a point cloud representing the patient at that time. As another example, tomography or other three-dimensional (3D) processing is used to form a 3D scene from multiple two-dimensional (2D) images (e.g., RGB images from different perspectives used to determine depth of imaged points). In another example, multiple 2D images are stitched together to form another 2D image with a larger field of view. The resulting 2D image can be used in subsequent processing.

The image or images are captured for a given time. Using continuous or defined frequency of capture, a stream of images from different times may be captured. The defined frequency may be preset, adjustable, or variable. By capturing images over time, camera data or images form a video of the patient. In alternative embodiments, an image or images for just one time are captured.

In act 104, an image processor detects breathing cycle information for the patient from the image or images. The image or images at a given time and/or images from different times are used to detect breathing cycle information, such as a breathing pattern. For example, a current or predicted breathing phase and/or breathing magnitude for the patient are detected from the image or images for the current time or the current time and past times.

The detection is from the RGB, thermal, depth, and/or other components of the captured image. For example, RGB, depth, or thermal information alone are used to detect the breathing.

The breathing cycle information may be the breathing phase and/or magnitude. For example, the instantaneous breathing phase is detected as one of inhalation, exhalation, or breath hold. As another example, the phase is detected with greater resolution, such as one of ten or more phase points through the breathing cycle (e.g., [0, 1] with 0.1 increments where beginning inhalation is 0 and beginning exhalation is 0.5). The magnitude may be an estimate of lung volume, height of chest or abdomen relative to a fixed reference (e.g., patient bed), and/or diaphragm position.

In act 104-2, the processor detects the breathing pattern and tracks the breathing pattern. The breathing pattern may be the cycle, such as determining phase and/or magnitude over time. This may allow for calculation of cycle length and/or other characteristics of the breathing cycle in addition to phase or magnitude. The breathing pattern may be an instantaneous combination of different information, such as a current or future phase and magnitude. The patient's breathing phase and magnitude are a breathing pattern for the current time frame and/or in one or more future time frames.

For prediction of breathing information at a future time, an instantaneous current breathing or series of past breathing are detected. For example, a cycle or part of a cycle of phase and/or magnitude are determined. The cycle may be fit to a model or used to estimate the breathing at the future time. For example, the breathing information for a time 0.1 seconds in the future is predicted. The timing (i.e., how far in the future) may relate to the operation of the scanner and/or communications with the scanner for control.

In one embodiment, a current breathing pattern is compared with a reference breathing pattern. The reference may be generalized or population-based reference. Alternatively, the reference is from a planning stage and is a measure from the patient. Where the detected breathing differs from the reference by a threshold amount (e.g., in length of cycle or magnitude), then the scanner is not operated.

The entire image may be used to detect the breathing information. In an alternative embodiment, one or more landmarks and/or regions of interest are detected in the image. The detection may be by processing, such as random walker or edge detection, or may be by application of a machine-learned model. For example, a region of the chest and/or abdomen having a greatest variation with breathing is identified. As another example, a mouth or nose region associated with greater variance in temperature due to breathing is identified. The detection of breathing information is then limited to the region and/or landmarks of interest. The breathing information is detected for a given time based on the region or regions. In one embodiment, the breathing information (e.g., phase) is detected at different times based on the region or regions of interest. The region and breathing are tracked over time. The patient's breathing pattern is regularly or continuously tracked throughout the scanning/treatment session. For each time, the breathing information is detected using the current image or current image and past images.

The detection uses image processing. For example, a magnitude over time is plotted. A curve is fit to the plot. The curve represents the cycle in magnitude, from which phase may be determined. As another example, a magnitude is matched via look-up table to a breathing phase. The signal processing algorithm processes the patient camera sensor data and outputs the breathing pattern.

In another embodiment, a machine-learned model detects. The current image or images are input to the model. Previous images may be input as well. Alternatively, or additionally, information derived from the images (e.g., average RGB value or depth for a region of interest) are input. The machine-learned model is trained to output breathing information in response to the input. A neural network, support vector machine, or Bayesian inference model may be used.

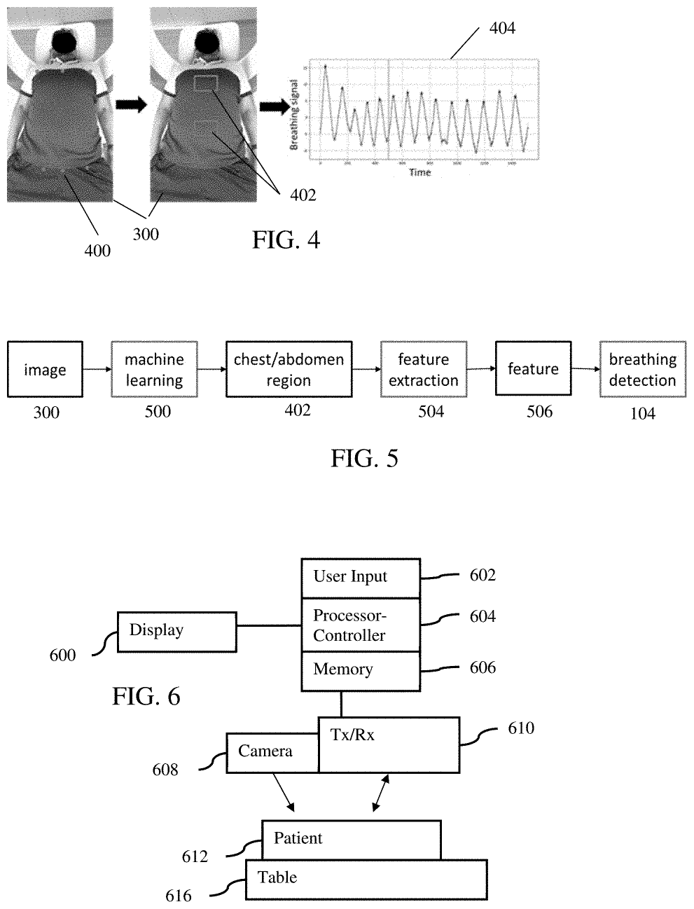

FIG. 4 illustrates an example embodiment. Landmarks 400 and/or regions of interest 402 are identified and used to detect breathing. The detection of the breathing cycle and/or current breathing phase uses values of features from the image 300, such as values for the landmarks 400 and/or regions 402. The breathing pattern 404 is detected from patient RGB, depth, or infrared image 300. The breathing pattern 404 is determined through monitoring the motion of patient's chest and/or abdomen region 402, such as being magnitude over time. Due to lung volume change during exhalation or inhalation, the patient's chest and/or abdomen region 402 moves up or down accordingly. Such motion can be captured in patient's RGB color, depth, or infrared image 300. Using a signal processing algorithm and/or machine learning algorithm, the information captured in the camera image 300 can be extracted to predict the breathing pattern 404.

FIG. 5 shows a further embodiment of the detection in act 104 of the breathing from the image 300. In this example, a sequence of RGBD images 300 from one depth camera are used to predict the breathing pattern 404. One or more (e.g., two) regions of interest 402 in the image 300 are identified. The patient's chest and abdomen regions 402 are automatically detected from each patient RGBD image 300 using a machine-learned model 500 (e.g., convolutional neural network trained with a machine learning algorithm). The input to the machine-learned model 500 is the RGBD or infrared data, and the output is the coordinates of the regions of interest 402 or coordinates of patient body landmarks 400, which are further used to determine the regions of interest 402. Values of features (i.e., feature values 506) are extracted in act 504 for each region 402. The feature of these regions can be any or combinations of the depth value, the RGB value, deep learning features (e.g., features output by a machine-learned model or used by a machine-learned model), and/or values derived from the camera data. In act 104, the breathing cycle information is detected from the feature value or values 506. Using the extracted features as the input, prediction is achieved through another machine-learned model (e.g., neural network, regression model, or classification model trained with a machine learning algorithm). Alternatively, the prediction is achieved through a non-learning method, such as a Principal Component Analysis. The prediction is a current state of breathing (e.g., current phase) or a future state.

The format of the prediction results depends on the need of the application. For example, the prediction is breathing phase information, such as inhalation/exhalation/hold phase. As another example, the phase is a continuous value between [−n, n], where n is any real number that is proportional to chest volume measurement and indicates the minimum and maximum volume. In yet another example, the prediction result can be proportional to the speed of the change of the chest volume change. In the example of FIG. 4, the prediction is output as a current or expected point along a cycle waveform where the waveform shows past magnitude of the feature value (e.g., average depth from the two regions 402).

In another embodiment, the camera data includes temperature information. For example, a thermal or infrared camera captures images with temperature information for the patient, such as at the head of the patient as the outer surface. The breathing pattern is detected through a patient thermal image. Breathing motion is captured using the thermal camera. By pointing the camera towards the head of the patient, the thermal camera captures the air flow caused by the patient breathing. Such information can be further used to determine the breathing pattern. For instance, using a patient thermal image, the head region of the patient is identified using a machine-learned model. The input to the model is the thermal image or an aligned RGB or RGBD image, and the output of the model is the coordinate of the head region in the infrared image. These coordinates are used to extract values for one or more features from the infrared image. For example, the thermal value of the region, a deep learning feature of the region, a statistical value of the thermal value of the region, or combinations thereof are extracted. Using these feature values as input, the breathing pattern (e.g., cycle or phase) is detected through a machine-learned model, such as a regression or classification model, or through a non-learning-based algorithm, such as a Principal Component Analysis.

In an alternative embodiment to identifying regions and extracting features, the breathing pattern is directly predicted by feeding the image or images (e.g., thermal image) and other corresponding sensor data to a machine-learned model. The machine-learned model is trained to predict the breathing pattern directly from the input images.

Referring again to FIG. 1, in act 106, the image processor controls a medical scanner with the breathing cycle information. The processor as a controller or through a controller controls the scanning. For example, the processor sends trigger or gating signals to the scanner. The scanner includes a port or interface for receiving trigger signals. As another example, a medical scanner internal command is provided for starting the scan (e.g., image acquisition cycle or treatment radiation cycle). In response, the scanner starts or stops scanning.

In one embodiment, the scanning of act 107 by the medical scanner is triggered. The breathing information is used to trigger scanning at particular times or phases in the breathing cycle. Given the predicted breathing pattern (e.g., current or future breathing phase), the medical scanner is automatically triggered. For instance, a rule-based mechanism triggers the image acquisition cycle or treatment cycle when the current breathing phase reaches inhalation phase and stops the image or treatment cycle when the patient starts exhaling. As another instance, the medical scanner is triggered to scan at a given phase of each breathing cycle. To avoid motion artifact, the medical scanner is controlled to scan (transmit and/or receive) only during one of inhalation, exhalation, or breath hold and/or at the same point in the breathing cycle. The phase, magnitude, or other breathing information are used to trigger the medical scanner to scan at a particular time or period. In one embodiment, the current or future phase and/or breathing cycle are predicted. The triggering is based on the breathing phases at different times in the cycle so that the medical scanner scans only during inhalation, exhalation, or breath hold.

The trigger is to begin and/or end the scanning. For example, imaging or radiation therapy may be started at one point in the breathing cycle and ended at another point in the same breathing cycle. By detecting the breathing phase in an on-going manner, the start and end of the scan are controlled to avoid motion problems. For another instance, a rule-base program triggers the image acquisition cycle or treatment cycle when the patient starts holding their breath and stops when the patient stops holding their breath. For another instance, a program compares the current breathing pattern (e.g., cycle or phase) to a reference breathing pattern (e.g., cycle or phase) acquired prior to the current scan. Where the patterns do not match (e.g., more than a threshold difference), then scanning is not triggered to ensure the consistency between each image acquisition and/or treatment.

FIG. 6 shows one embodiment of a medical scanner. The medical scanner includes a display 600, user input 602, memory 606, and image processor 604. The medical scanner also includes the camera 608 for sensing (imaging) an outer surface of a patient 612 and transmitter and/or receiver 610 for scanning the patient 612. The display 600, image processor 604, and memory 606 may be part of a medical imaging or therapy system (e.g., part of the transmitter and/or receiver 610) or may be a computer, server, workstation, or another system for image processing communicatively connected with the medical imaging or therapy system.

Additional, different, or fewer components may be provided. For example, a computer network is included for remote breathing detection and/or control of the transmitter and/or receiver 610. As another example, one or more machine-learned models are stored in the memory 606 and applied by the processor 604 to detect breathing. In yet another example, the transmitter and/or receiver 610 includes a trigger interface communicatively connected with the processor 604.

The camera 608 images the patient 612. The camera 608 is a depth sensor, optical camera, 3D camera, infrared camera, thermal camera, and/or another type of camera. LIDAR, 2.5D, RGBD, stereoscopic optical sensor, or other depth sensor may be used. The camera 608 may include a separate processor for determining depth measurements from images and/or detecting objects represented in images, or the image processor 604 determines the depth measurements from images captured by the camera 608. The depth may be relative to the camera 608 and/or a bed or table 616. Alternatively, a camera without depth sensing is used. One camera 608 is shown, but multiple cameras may be used, such as viewing the patient 612 on the bed or table 616 from different angles and/or distances. A light projector may be provided. The camera 608 may directly measure depth from the camera 608 to the patient.

The camera 608 is directed to the patient 612. The camera 608 may be part of or connected to the transmitter and/or receiver 610 (e.g., connected to a housing, gantry, arm (e.g., C-arm), probe, or another part of the medical imaging or therapy system. In one embodiment, one or more cameras 608 are positioned on the ceiling and/or walls.

The transmitter and/or receiver 610 is the part of the scanner that scans the patient. For example, an x-ray source transmits x-rays for treatment or imaging. In imaging, a detector detects the x-rays after passing through the patient 612. As another example, a gamma camera or emissions detector detects emissions from a radiopharmaceutical in the patient (i.e., single photo emission computed tomography or positron emission tomography detector). In yet another example, a beamformer and probe scans with ultrasound for therapy and/or imaging. As another example, a pulse generator, coils, and magnetic resonance receiver scan as a magnetic resonance system. The transmitter and/or receiver 610 are for scanning to apply therapy or scanning to image the patient 612.

The user input 602 is configured, through a user interface operated by the image processor 604 or another processor, to receive and process user input. The user input 602 is a device, such as keyboard, button, slider, dial, trackball, mouse, or another device. The user input 602 may but does not need to receive scanning triggers from the user since the scan triggering is automated based on images from the camera 608.

The image processor 604 is a control processor (e.g., controller), general processor, digital signal processor, three-dimensional data processor, graphics processing unit, application specific integrated circuit, field programmable gate array, artificial intelligence processor, digital circuit, analog circuit, combinations thereof, or another now known or later developed device for image processing to detect breathing. The image processor 604 is a single device, a plurality of devices, or a network. For more than one device, parallel or sequential division of processing may be used. Different devices making up the image processor 604 may perform different functions, such as detecting breathing by one device and generating a trigger signal by another device. In one embodiment, the image processor 604 is a control processor or other processor of a medical scanning system (e.g., of the transmitter and/or receiver 610). The image processor 604 operates pursuant to and is configured by stored instructions, hardware, and/or firmware to perform various acts described herein.

The image processor 604 is configured to detect breathing of the patient from camera data from the camera 608. Any breathing information, such as a phase or cycle is detected. Data from the camera 608 is used to detect. For example, one or more landmarks or regions are detected from RGB data. Depths for the landmarks or regions at a given time or over time are used to determine the phase and/or cycle. In one embodiment, the processor 604 detects the breathing by identification of a region of interest, extraction of a value for a feature of the region of interest from the camera data, and detection of the breathing from the value. The processor 604 may apply a signal processing program and/or one or more machine-learned models to detect the breathing from the image (camera data) or features derived from the image.

The processor 604 is a controller. In alternative embodiments, a separate controller is provided. The processor 604 communicates with the separate controller, and the controller activates and/or deactivates scanning by the transmitter and/or receiver 610. The controller is configured to trigger scanning of the patient 612 by the transmitter or receiver 610. The timing of the trigger to start or stop scanning is based on the detected breathing. For example, the transmitter and/or receiver 610 is turned on and/or off so that the scan occurs only during inhalation, exhalation, or breath hold or occurs only at particular phases of the breathing.

The display 600 is a CRT, LCD, projector, plasma, printer, tablet, smart phone, or another now known or later developed display device for displaying detected breathing information, such as the cycle or current phase. The display 600 may display scan information, such as a medical image or treatment course. By using automated control of the scan based on detected breathing, the imaging and/or therapy are less likely subjected to inaccuracies due to movement of the patient in breathing.

The camera data (images or surface data), landmarks, regions, machine-learned model, breathing information, and/or other information are stored in a non-transitory computer readable memory, such as the memory 606. The memory 606 is an external storage device, RAM, ROM, database, and/or a local memory (e.g., solid state drive or hard drive). The same or different non-transitory computer readable media may be used for the instructions and other data. The memory 606 may be implemented using a database management system (DBMS) and residing on a memory, such as a hard disk, RAM, or removable media. Alternatively, the memory 606 is internal to the processor 604 (e.g., cache).

The instructions for implementing the methods, processes, and/or techniques discussed herein are provided on non-transitory computer-readable storage media or memories, such as a cache, buffer, RAM, removable media, hard drive, or other computer readable storage media (e.g., the memory 606). Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code, and the like, operating alone or in combination.

In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. Because some of the constituent system components and method steps depicted in the accompanying figures may be implemented in software, the actual connections between the system components (or the process steps) may differ depending upon the manner in which the present embodiments are programmed.

Various improvements described herein may be used together or separately. Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

What is claimed is:

1. A method for camera-based respiratory triggered medical scanning, the method comprising:
capturing, with a camera, an image of an outer surface of a patient;
detecting, by a processor, breathing cycle information for the patient from the image; and
controlling, by the processor, a medical scanner with the breathing cycle information.

2. The method of claim 1 wherein capturing comprises capturing with a depth camera, and wherein detecting comprises detecting based, at least in part, on depth information from the depth camera.

3. The method of claim 1 wherein capturing comprises capturing with an infrared or thermal camera, and wherein detecting comprises detecting from temperature information at a head of the patient as the outer surface.

4. The method of claim 1 wherein capturing comprises capturing with the camera connected to the medical scanner.

5. The method of claim 1 wherein capturing comprise capturing the image with the camera and an additional image with an additional camera, and wherein detecting comprises detecting from the image and the additional image.

6. The method of claim 1 wherein detecting comprises determining values of features from the image and detecting the breathing cycle information from the values.

7. The method of claim 1 wherein detecting the breathing cycle information comprises detecting the breathing phase and/or magnitude.

8. The method of claim 1 wherein detecting comprises detecting the breathing cycle information as a prediction of breathing at a future time.

9. The method of claim 1 wherein detecting comprises detecting the breathing cycle information as an output of a machine-learned model in response to input of the image to the machine-learned model.

10. The method of claim 1 wherein controlling comprises triggering scanning by the medical scanner.

11. The method of claim 1 wherein capturing, detecting, and controlling are performed free of input from a breath sensor and free of input from a breathing sensor.

12. The method of claim 1 wherein detecting comprises identifying a region of interest in the image, extracting a feature value for the region of interest in the image, and detecting the breathing cycle information from the feature value.

13. The method of claim 1 wherein the breathing cycle information comprises a phase of the breathing cycle as inhalation, exhalation, or breath hold, and wherein controlling comprises triggering the medical scanner to scan only during one of inhalation, exhalation, or breath hold.

14. A medical scanner comprising:
a camera configured to image a patient;
an image processor configured to detect breathing of the patient from camera data from the camera;
a transmitter or receiver configured to scan the patient;
a controller configured to trigger scanning of the patient by the transmitter or receiver, timing of the trigger based on the detected breathing.

15. The medical scanner of claim 14 wherein the camera is a depth camera, thermal camera, or infrared camera, and wherein the transmitter or receiver is an imager or a therapy device.

16. The medical scanner of claim 14 wherein the image processor is configured to detect the breathing as a breathing cycle phase, and wherein the controller is configured to turn on and/or off the transmitter or receiver such that the scan occurs only during inhalation, exhalation, or breath hold.

17. The medical scanner of claim 14 wherein the image processor is configured to detect the breathing by identification of a region of interest, extraction of a value for a feature of the region of interest from the camera data, and detect the breathing from the value.

18. The medical scanner of claim 14 wherein the image processor is configured to detect by application of a machine-learned model to the camera data.

19. A method for camera-based respiratory triggered medical scanning, the method comprising:
capturing, with a camera, an image of a patient;
detecting, by a processor, breathing phase or magnitude for the patient from the image; and
triggering a medical scanner to scan at a time based on the breathing phase or magnitude.

20. The method of claim 19 wherein capturing comprises capturing over time with the camera being a depth camera, thermal camera, RGB camera, or an infrared camera, wherein detecting comprises detecting the breathing phases at different times based on a region of interest, and wherein triggering comprises triggering the medical scanner to scan only during inhalation, exhalation, or breath hold based on the breathing phases at the different times.
